(19) United States
(12) Patent Application Publication
(10) Pub. No.: US 2011/0009520 A1
(43) Pub. Date: Jan. 13, 2011

(54) DIMENSIONALLY STABLE, SHAPED ARTICLES COMPRISED OF DRIED, AGGREGATED, WATER-SWELLABLE HYDROGEL MICROSPHERES AND METHOD OF MAKING SAME

(76) Inventors: Garret D. Figuly, Wilmington, DE (US); Surbhi Mahajan, Newark, DE (US)

Correspondence Address: E I DU PONT DE NEMOURS AND COMPANY;LEGAL PATENT RECORDS CENTER, BARLEY MILL PLAZA 25/1122B, 4417 LANCASTER PIKE, WILMINGTON, DE, 19805, US

(21) Appl. No.: 12/919,558

(22) PCT Filed: Mar. 19, 2009
(86) PCT No.: PCT/US2009/037599

§ 371 Date: Aug. 26, 2010

Related U.S. Application Data
(60) Provisional application No. 61/070,201, filed on Mar. 20, 2008.

(51) Int. Cl. A61L 27/52

(52) U.S. Cl. 523/113

(57) ABSTRACT

Dimensionally stable, shaped articles comprised of dried, aggregated, water-swellable hydrogel microspheres are described. The microspheres are aggregated together without the use of a binding agent. When exposed to an aqueous medium in a container, the dimensionally stable, shaped article swells slowly and disaggregates, forming at least partially swollen hydrogel microspheres, which take the shape of the container. The dimensionally stable, shaped articles disclosed herein have many potential applications, including medical applications such as a medical implant.

### CROSS-REFERENCE TO RELATED APPLICATION

This application claims priority under 35 U.S.C. §119 from U.S. Provisional Application Ser. No. 61/070201, filed Mar. 20, 2008.

### FIELD OF INVENTION

The invention relates to hydrogel microspheres. More specifically, the invention relates to dimensionally stable, shaped articles comprised of dried, aggregated, water swellable microspheres. The shaped articles have many potential applications, including use as medical implants.

### BACKGROUND OF THE INVENTION

Hydrogel microspheres have many potential applications, including medical applications. For example, microspheres with high density, yet a large capacity to swell in an aqueous environment, are useful for absorption applications such as small-scale spill control and for delivery applications in which they carry and release active ingredients such as fertilizers, herbicides, pesticides, cosmetics, and shampoos. Medical applications of hydrogel microspheres include tissue augmentation, void filling, wound treatment, embolization, and drug delivery. Tissue augmentation involves introduction of materials in a collapsed area to provide a filling function, such as the treatment of scars or wrinkles. Void filling involves introduction of materials into an empty space, such as one created by removal of a tissue mass. Wound treatment involves introduction of materials to stop bleeding, provide padding, deliver medication, and absorb fluids. Such materials are useful especially in emergency situations including accidents and military operations. Embolization treatment involves the introduction of a material into the vasculature in order to block the blood flow in a particular region, and may be used to treat non-cancerous tumors, such as uterine fibroids, and cancerous tumors, as well as to control bleeding caused by conditions such as stomach ulcers, aneurysms, and injury. Blockage may be desired in the case of arteriovenous malformation (AVM), where abnormal connections occur between arteries and veins. Additionally, blockage may be desired for pre-surgical control of blood flow.

Various types of water-swellable hydrogel microspheres and methods of use have been developed to meet the needs of the aforementioned applications (see for example, Vogel et al., U.S. Pat. No. 6,436,424, Vogel et al., U.S. Pat. No. 6,660,301, and Figuly et al., copending and commonly owned U.S. Patent Application Publication Nos. 2007/0237956, 2007/0237742, 2007/0237830, and 2007/0237741). The microspheres are used as a powder or as a suspension in a carrier medium. However, for some applications it would be highly desirable to have a dimensionally stable, shaped article comprised of dried, water-swellable hydrogel microspheres, which would be easier to handle and deliver than individual microspheres, for example a medical implant.

Therefore, the problem to be solved is to provide water-swellable hydrogel microspheres in the form of a dimensionally stable article which can be formed in a variety of shapes. The stated problem is addressed herein by the discovery of a method of making dimensionally stable, shaped articles from water-swellable hydrogel microspheres which can be made into any desired shape.

### SUMMARY OF THE INVENTION

In various embodiments, the invention provides dimensionally stable, shaped articles comprising dried, water-swellable hydrogel microspheres that are aggregated together to form a predetermined shape. The invention also provides a method of making the dimensionally stable, shaped articles disclosed herein. Additionally, the invention provides a method for completely or partially blocking or filling a lumen or void within the body of a mammal using the dimensionally stable, shaped articles disclosed herein.

Accordingly in one embodiment, the invention provides a dimensionally stable, shaped article comprising dried, water-swellable hydrogel microspheres, aggregated to form a predetermined shape, wherein said article does not contain a binding agent to bind the microspheres together.

In another embodiment, the invention provides a method of making a dimensionally stable, shaped article comprised of dried, aggregated, water-swellable hydrogel microspheres comprising the steps of:
a) providing in a mold having a preselected shape, a suspension of water-swellable hydrogel microspheres in an aqueous medium wherein said microspheres are at least partially swollen; and
b) evaporatively drying said suspension to remove substantially all of the aqueous medium to form the dimensionally stable, shaped article;
wherein:
   (i) said method is carried out in the absence of a binding agent; and
   (ii) said dimensionally stable shaped article has the shape of the mold.

In another embodiment, the invention provides a dimensionally stable, shaped article prepared by a process comprising the steps of:
a) providing in a mold having a preselected shape, a suspension of water-swellable hydrogel microspheres in an aqueous medium wherein said microspheres are at least partially swollen; and
b) evaporatively drying said suspension to remove substantially all of the aqueous medium to form the dimensionally stable, shaped article;
wherein:
   (i) said process is carried out in the absence of a binding agent; and
   (ii) said dimensionally stable shaped article has the shape of the mold.

In another embodiment, the invention provides a method for completely or partially blocking or filling a lumen or void within the body of a mammal comprising the steps of:
a) providing a dimensionally stable, shaped article comprised of dried, water-swellable hydrogel microspheres, aggregated to form a predetermined shape, wherein said article does not contain a binding agent to bind the microspheres together;
b) implanting the dimensionally stable, shaped article into a lumen or void within the body; and
c) allowing the dimensionally stable, shaped article to swell and disaggregate upon exposure to a physiological aqueous fluid present in or surrounding the lumen or void, thereby forming at least partially swollen hydrogel microspheres, which totally or partially block or fill the lumen or void.

### BRIEF DESCRIPTION OF THE FIGURES

FIGS. 1a (low magnification) and 1b (high magnification) show electron micrographs of a disk-shaped pellet comprised of dried, aggregated water-swellable hydrogel microspheres prepared from 95% acrylic acid and 5% 2-hydroxyethyl methacrylate, as described in Example 1.

FIGS. 2a (low magnification) and 2b (high magnification) show electron micrographs of a disk-shaped pellet comprised of dried, aggregated water-swellable hydrogel microspheres prepared from 95% acrylic acid and 5% 2-hydroxyethyl acrylate, as described in Example 2.

FIG. 3 is a picture of various shaped articles prepared as described in Examples 6-14. The number above each shape corresponds to the Example number that describes the article. A United States penny is included in the picture for size comparison.

### DETAILED DESCRIPTION OF THE INVENTION

Disclosed herein are dimensionally stable, shaped articles comprising dried, water-swellable hydrogel microspheres that are aggregated together to form a predetermined shape. Also disclosed herein is a method of making the shaped articles. The article can be made in virtually any shape by the use of an appropriate mold.

The dimensionally stable, shaped articles disclosed herein have many potential applications, including medical applications such as a medical implant for tissue augmentation, void filling, wound treatment, spinal disk reconstruction, joint repair, embolization, drug delivery, as a plug for the punctum to treat dry eye syndrome, as a plug to seal a fistula, and as a plug to treat urinary incontinence. Additionally, the dimensionally stable, shaped articles disclosed herein may be useful for other applications including, but not limited to, absorption applications, such as small-scale spill control; and delivery applications to carry and release active ingredients such as a spike that may be inserted into the ground to release fertilizers, herbicides, and pesticides.

The following definitions and abbreviations are to be used for the interpretation of the claims and the specification.

The term “microspheres” or “microsphere” refers to either a population of micron size particles, or an individual particle, depending upon the context in which the word is used, which has a high sphericity measurement. The sphericity measurement of a population of microspheres may be in the range of about 80% to about 100%, with 95% being typical. The microspheres are substantially spherical, although a microsphere population may include some individual particles that have a lower sphericity measurement.

The term “water-swellable hydrogel microspheres” refers to microspheres which are substantially water-insoluble and are capable of absorbing a substantial amount of water, thereby increasing in volume when contacted with water or an aqueous medium.

The term “miscible” refers to the ability of two liquids to mix without separating into two separate phases. In addition, a solid is miscible if a solution made with the solid is miscible with another liquid. Specifically, a liquid monomer may itself be miscible with water. A solid monomer is water miscible when an aqueous solution prepared with the solid monomer can be mixed with water without having the mixture separate into two separate phases.

The term “substantially chlorinated hydrocarbon” refers to a hydrocarbon that is from 50% to fully chlorinated. Carbon tetrachloride is an example of such a hydrocarbon.

The term “slurry” refers to a composition that is a particulate material in a liquid.

The terms “first suspension” and “second suspension” refer to suspensions formed during a process of preparing microspheres that is described herein.

The term “evaporatively drying” as used herein, refers to the slow removal of the aqueous medium in which the water-swellable hydrogel microspheres are suspended. The removal of the aqueous medium may be done passively at ambient conditions of temperature and humidity. Additionally, the removal of the aqueous medium may be done under controlled conditions of temperature and humidity at which the rate of water removal is comparable to the rate at ambient conditions.

The term “medical implant” as used herein, refers to a dimensionally stable, shaped article, which may be implanted in the body of a mammal for medical applications including, but not limited to, tissue augmentation, void filling, wound treatment, spinal disk reconstruction, joint repair, embolization, drug delivery, as a plug for the punctum to treat dry eye syndrome, as a plug to seal a fistula, and as a plug to treat urinary incontinence. The medical implant disclosed herein is comprised of dried, aggregated, water-swellable hydrogel microspheres. When implanted into a lumen or void within the body, the implant swells and disaggregates upon exposure to a physiological aqueous fluid present in or surrounding the lumen or void, thereby forming at least partially swollen hydrogel microspheres, which totally or partially block or fill the lumen or void.

The term “lumen” as used herein, refers to any hollow organ or vessel of the body of a mammal, including, but not limited to, fallopian tubes, ureter, vas deferens, veins, arteries, intestine, trachea, punctum (i.e., tear drainage duct), and the like.

The term “void” as used herein, refers to any hollow space created by congenital abnormalities, disease, aging, and/or surgery such as extraction of tumors and other growth masses. As such, the term “void” includes, but is not limited to, lesions, fissures, fistulae, cysts, diverticulae, aneurysms, and the like.

The meaning of abbreviations used is as follows: “min” means minute(s), “h” means hour(s), “sec” means second(s), “μL” means microliter(s), “mL” means milliliter(s), “L” means liter(s), “nm” means nanometer(s), “mm” means millimeter(s), “cm” means centimeter(s), “μm” means micrometer(s) or micron(s), “mM” means millimolar, “M” means molar, “g” means gram(s), “mol” means mole(s), wt %” means percent by weigh, “AA” means acrylic acid, “HEMA” means 2-hydroxylethyl methacrylate, “HEA” means 2-hydroxylethyl acrylate.

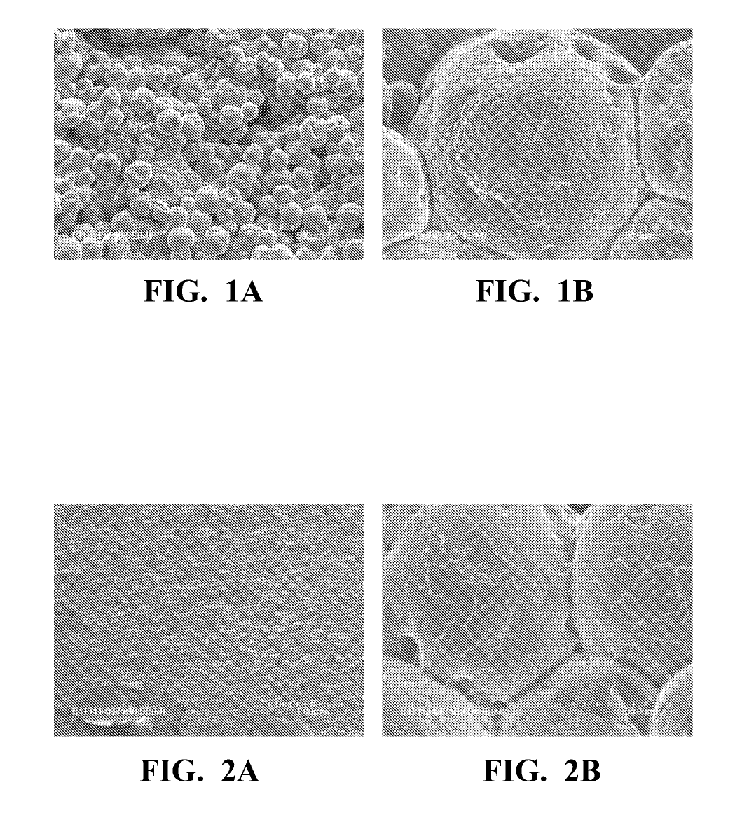

When a suspension of water-swellable hydrogel microspheres in an aqueous medium is dried under conditions described in the art, e.g., drying under vacuum in a vacuum oven at about 20° C. to about 100° C. with a nitrogen purge, the microspheres form a free-flowing powder (Figuly et al, copending and commonly owned U.S. Patent Application Publication No. 2007/0237956). However, it was unexpectedly discovered that when a suspension of water-swellable hydrogel microspheres in an aqueous medium is evaporatively dried in a container, the microspheres aggregate together, without the use of a binding agent, to form a dimensionally stable article that retains the shape of the container. When formed at the proper conditions, as described herein below, the dimensionally stable article can be handled without breaking apart. While not wishing to be bound by any particular theory, it is speculated that the evaporative drying allows the polymer chains on the surface of the microspheres to become intertwined, thereby partially integrating the surface of the microspheres together to form the aggregated shape (see the electron micrographs in FIGS. 1a and 1b and 2a and 2b, which are described in Examples 1 and 2, respectively). When the dimensionally stable, shaped article comprised of the dried, aggregated, water-swellable hydrogel microspheres is placed in an aqueous medium in a container (e.g., a lumen or void within the body of a mammal), the article swells slowly and disaggregates, forming at least partially swollen hydrogel microspheres, which take the shape of the container. In contrast, individual water-swellable hydrogel microspheres swell very rapidly, as described below.

### Water-Swellable Hydrogel Microspheres

The microspheres suitable for use in the process disclosed herein are water-swellable hydrogel microspheres, which comprise various polymers that are typically crosslinked, although uncrosslinked polymers may also be used. The polymer composition of the hydrogel microspheres may be chosen from a wide variety of polymers known in the art depending on the intended application. Examples of polymer compositions of the hydrogel microspheres include, but are not limited to, polymers comprising at least one monomer selected from the group consisting of acrylic acid, methacrylic acid, salts (such as sodium and ammonium) of acrylic and methacrylic acid, acrylamide, methacrylamide, N-substituted acrylamides, N-substituted methacrylamides, vinyl alcohol, vinyl acetate, methyl maleate, 2-acryloylethane-sulfonic acid, 2-methacryloylethane-sulfonic acid, salts of 2-acryloylethane-sulfonic acid and 2-methacryloylethane-sulfonic acid, styrene-sulfonic acid, salts of styrene-sulfonic acid, 2-hydroxyethyl acrylate, 2-hydroxyethyl methacrylate, isobutylene, maleic anhydride, acrylonitrile, and ethylene glycol. Other suitable polymers include saponification products of copolymers of vinyl acetate and acrylic acid ester, vinyl acetate and acrylic acid ester copolymer, vinyl acetate and methyl maleate copolymer, isobutylene-maleic anhydride crosslinked copolymer, starch-acrylonitrile graft copolymer and its saponification products, and crosslinked polyethylene oxide. Most useful hydrogel microspheres for medical applications comprise monomers having biocompatibility such as acrylic acid, methacrylic acid, salts of acrylic acid and methacrylic acid, 2-hydroxyethyl acrylate and 2-hydroxyethyl methacrylate, and combinations thereof.

In one embodiment, the polymer composition of the water-swellable, hydrogel microspheres is a combination comprising acrylic acid and at least one monomer from the group consisting of sodium acrylate, 2-hydroxyethyl methacrylate, 2-hydroxyethyl acrylate, styrene sulfonic acid and salts of styrene sulfonic acid.

In another embodiment, the polymer composition of the water-swellable, hydrogel microspheres comprises styrene sulfonic acid or a combination comprising styrene sulfonic acid and the sodium salt of styrene sulfonic acid.

In another embodiment, the polymer composition of the water-swellable, hydrogel microspheres comprises acrylic acid, sodium acrylate and vinyl alcohol.

The water-swellable, hydrogel microspheres may be prepared using methods known in the art, such as those described by Kitagawa (U.S. Pat. No. 6,218,440), Vogel et al. (U.S. Pat. No. 6,218,440), Hori et al. (JP 06056676), Horak et al. (Biomaterials 7:188-192, 1986), and Lewis et al. (U.S. Patent Application Publication No. 2006/0204583). In one embodiment, the water-swellable, hydrogel microspheres are prepared by the method described by Figuly et al. (U.S. Patent Application Publication No. 2007/0237956), as described in detail below.

Monomer and Crosslinking Agent

Monomers that may be used in the process described by Figuly et al. supra, for preparing water-swellable hydrogel microspheres are water miscible monomers including, but not limited to, acrylic acid, methacrylic acid, salts (such as sodium and ammonium) of acrylic acid and methacrylic acid, acrylamide, methacrylamide, N-substituted acrylamides, N-substituted methacrylamides, 2-acryloylethane-sulfonic acid, 2-methacryloylethane-sulfonic acid, salts of 2-acryloylethane-sulfonic acid and 2-methacryloylethane-sulfonic acid, styrene-sulfonic acid, salts of styrene-sulfonic acid, 2-hydroxyethyl acrylate, and 2-hydroxyethyl methacrylate. Monomers may be used singly or in combinations as co-monomers. Monomers that perform well as single monomer components (subgroup 1) include acrylic acid, methacrylic acid, salts (such as sodium and ammonium) of acrylic acid and methacylic acid, 2-acryloylethane-sulfonic acid, 2-methacryloylethane-sulfonic acid, salts of 2-acryloylethane-sulfonic acid and 2-methacryloylethane-sulfonic acid, styrene-sulfonic acid, and salts of styrene-sulfonic acid. Preferably, the following monomers are used as co-monomers with at least one of the monomers from subgroup 1: acrylamide, methacrylamide, N-substituted acrylamides, N-substituted methacrylamides, 2-hydroxyethyl acrylate, and 2-hydroxyethyl methacrylate. Most useful in producing microspheres for medical applications are monomers having biocompatibility such as acrylic acid, methacrylic acid, salts of acrylic acid and methacylic acid, 2-hydroxyethyl acrylate and 2-hydroxyethyl methacrylate, and combinations thereof. In one embodiment the monomer is a combination comprising acrylic acid and at least one monomer from the group of sodium acrylate, 2-hydroxyethyl methacrylate, 2-hydroxyethyl acrylate, styrene sulfonic acid, and the sodium salt of styrene sulfonic acid.

In another embodiment, the monomer is styrene sulfonic acid or a combination comprising styrene sulfonic acid and the sodium salt of styrene sulfonic acid.

Many of these monomers are liquids which are miscible with water. For monomers that are solids, an aqueous solution of the monomer may be prepared, and this monomer solution is miscible with water. Acid monomers and salts of monomers may be combined to adjust the pH of a monomer solution. It is particularly useful to partially neutralize an acid monomer, thereby providing a mixture of acid monomer and monomer salt. Acid monomers that may be used are, for example, acrylic acid, methacrylic acid, 2-acryloylethane-sulfonic acid, 2-methacryloylethane-sulfonic acid, styrene-sulfonic acid, and combinations thereof. A monomer prior to partial neutralization is referred to as an initial monomer. An acid monomer is typically partially neutralized using a base. Suitable bases include, but are not limited to, sodium hydroxide, potassium hydroxide, ammonium hydroxide, lithium hydroxide and combinations thereof. Bases containing divalent cations, such as calcium hydroxide and barium hydroxide may also be used; however, they are preferably used in combination with a base containing monovalent cations because divalent cations have a strong tendency to induce ionic crosslinking, which could severely alter the desirable properties of the microspheres. For some applications it may be desirable to substitute a portion of the base with barium hydroxide (Ba(OH)₂) to introduce a radio-opaque element, which makes the resulting microspheres amenable to x-ray imaging. Barium hydroxide may be used in a ratio of up to about 1:1 by weight of Ba(OH)₂ to NaOH, to produce a combination salt that includes barium salt. Alternatively, a barium monomer salt may be included in a monomer combination.

A crosslinking agent that is miscible with an aqueous monomer solution is copolymerized with the monomer in the process described by Figuly et al., supra. Examples of crosslinking agents that may be used include, but are not limited to, N,N′-methylene-bis-acrylamide, N,N′-methylene-bis-methacrylamide, N-methylolacrylamide, N-methylolmethacrylamide, glycidyl acrylate, glycidyl methacrylate, polyethylene glycol diacrylate and polyethylene glycol dimethacrylate (which are most useful with hydrophobic monomers), polyvalent metal salts of acrylic acid and methacrylic acid, divinyl benzene phosphoacrylates, divinylbenzene, divinylphenylphosphine, divinyl sulfone, 1,3-divinyltetramethyldisiloxane, 3,9-divinyl-2,4,8,10-tetraoxaspiro[5,5]undecane, phosphomethacrylates, and polyol polyglycidyl ethers such as ethylene glycol diglycidyl ether, glycerin triglycidyl ether, glycerin diglycidyl ether, and polyethylene glycol diglycidyl ether, and combinations thereof. The amount of crosslinking agent included for copolymerization may vary and is inversely related to the amount of swell capacity in the microspheres produced using the process. The exact amount of crosslinking agent needed will vary depending on the specific agent used and can be readily determined by one skilled in the art. The amount of crosslinking agent is calculated as Mol % (mole percent) based on the sum of the moles of monomer and moles of crosslinking agent. Thus, the Mol % is calculated as moles of crosslinking agent/(moles of monomer+moles of crosslinking agent). Preferably, the Mol % of crosslinking agent is equal to or less than about 5 Mol %, preferably, equal to or less than about 4 Mol %, more preferably about 0.08 Mol % to about 4 Mol %, most preferably about 0.08 Mol % to about 2.3 Mol % relative to total moles of monomer and crosslinking agent.

First Solution

A monomer and crosslinking agent as described above are prepared in an aqueous solution, together with additional components, which is herein called the “first solution”. The monomer is generally included at about 0.5% to about 30% as weight percent of the first solution. Monomer weight percents of about 15% to about 25% and about 20% to about 25% are particularly useful in the process described by Figuly et al., supra. If a combination of monomers is used in the process, the total amount of all the monomers is about 0.5% to about 30%, in addition from about 15% to about 25%, and in addition from about 20% to about 25%, as weight percent of the first solution.

The pH of the first solution may vary and is a factor in the swell capacity of the microspheres prepared in the process described by Figuly et al. supra. The useful pH range of the first solution also depends on the particular monomer or combination of monomers used. If the first solution contains at least one monomer from subgroup 2 consisting of acrylic acid, methacrylic acid, salts of acrylic acid and methacylic acid, acrylamide, methacrylamide, N-substituted acrylamides, N-substituted methacrylamides, 2-hydroxyethyl acrylate, and 2-hydroxyethyl methacrylate, but does not contain a monomer from subgroup 3 consisting of 2-acryloylethane-sulfonic acid, 2-methacryloylethane-sulfonic acid, salts of 2-acryloylethane-sulfonic acid and 2-methacryloylethane-sulfonic acid, styrene-sulfonic acid, and salts of styrene-sulfonic acid, then the pH of the first solution is at least about 3, preferably between about 3.5 and about 10, more preferably between about 5 and about 9, to produce microspheres with a high swell capacity. For example, a mixture of acrylic acid and sodium acrylate at a pH of between about 3.5 and about 10, and a 2 to 5 Mol % of N,N′-methylenebisacrylamide crosslinking agent (with respect to the monomer), when used in the process of Figuly et al., supra, produces microspheres with a swell capacity of at least about 80 grams of water per gram of microspheres. If the first solution contains at least one monomer from subgroup 3 consisting of 2-acryloylethane-sulfonic acid, 2-methacryloylethane-sulfonic acid, salts of 2-acryloylethane-sulfonic acid and 2-methacryloylethane-sulfonic acid, styrene-sulfonic acid, and salts of styrene-sulfonic acid, then the pH of the first solution is less than about 3 to produce highly swellable microspheres.

The pH of the first solution may be adjusted in any number of ways. For example, if the monomer is prepared as a monomer solution, as described above, the pH of the monomer solution will govern the pH of the first solution. In the case of an acid monomer, the pH of the monomer solution is related to the amount of base or monomer salt added to the acidic monomer solution. Alternatively, the pH of the first solution may be adjusted as required by the addition of acid or base after all the components have been added.

Included in the “first solution” is a component that can modify the viscosity of an aqueous solution to provide a surface tension that allows droplet formation in the aqueous/organic suspension that is formed during the microsphere preparation process. This component is referred to herein as a “protecting colloid”. A variety of natural and synthetic compounds that are soluble in aqueous media may be used as a protecting colloid including cellulose derivatives, polyacrylates (such as polyacrylic acid and polymethacrylic acid), polyalkylene glycols such as polyethylene glycol, partially hydrolyzed polyvinyl alcohol and other polyols, guar gum, and agar gum. Particularly useful are cellulose ethers such as methyl cellulose, hydroxymethyl cellulose, hydroxyethyl cellulose, ethylhydroxyethyl cellulose, hydroxypropyl cellulose, ethyl cellulose, and benzyl cellulose; as well as cellulose esters such as cellulose acetate, cellulose butylate, cellulose acetate butylate, cellulose propionate, cellulose butyrate, cellulose acetate propionate, cellulose acetate butyrate, and cellulose acetate phthalate. The amount of the protecting colloid in the first solution is sufficient to reduce microdroplet coalescence in the aqueous/organic suspension, and is generally between about 0.1% and about 3% by weight % of the first solution. Preferred is methyl cellulose at about 0.5% to about 0.6% by weight.

An emulsifier is included in the first solution to promote the formation of a stable emulsion on addition of the first solution to an organic second solution (described below). Any emulsifier which stabilizes the aqueous/organic emulsion may be used. Suitable emulsifiers include, but are not limited to, alkylaryl polyether alcohols such as the Triton™ X nonionic surfactants commercially available from Union Carbide (Danbury, Conn.). These products generally contain mixtures of polyoxyethylene chain lengths and include, for example, Triton® X-100: polyoxyethylene(10) isooctylphenyl ether; Triton® X-100, reduced: polyoxyethylene(10) isooctylcyclohexyl ether; Triton® N-101, reduced: polyoxyethylene branched nonylcyclohexyl ether; Triton® X-114: (1,1,3,3-tetramethylbutyl)phenyl-polyethylene glycol; Triton® X-114, reduced: polyoxyethylene(8) isooctylcyclohexyl ether; Triton® X-405, reduced: polyoxyethylene(40) isooctylcyclohexyl ether; and Triton™ X-405: polyoxyethylene(40) isooctylphenyl ether, 70% solution in water. Particularly suitable is Triton™ X-405, 70 wt % solution, which is an alkylaryl polyether alcohol preparation having an average of at least about 30 ethylene oxide units per ether side chain. Typically, the emulsifier in the first solution is used at a concentration of about 1% to about 10% by weight % of the first solution.

In addition, the first solution includes a polymerization initiator. The initiator used in the process of Figuly et al., supra is a water soluble azo initiator which has a low temperature of activation. Azo initiators are substituted diazo compounds that thermally decompose to generate free radicals and nitrogen gas. The temperature of activation of the azo initiator used is low enough so that the boiling point of an organic second solution (described below) is above the azo initiator activation temperature. Examples of suitable low temperature water soluble azo initiators include, but are not limited to, 2,2′-azobis(2-amidinopropane)dihydrochloride; 4,4′-azobis(4-cyanopentanoic acid); and 2,2′-azobis(2-[2-imidazolin-2-yl])propane dihydrochloride. A particular azo initiator, having a particular activation temperature, is used with an organic second solution composition (described below) at a temperature and with a reaction time period that is effective in initiating polymerization. Most effective is use of an azo initiator at a temperature that is close to its optimal activation temperature and which is also below the boiling temperature of the organic second solution. However, an azo initiator may be used at a temperature that is lower than its optimal activation temperature in order to stay below the boiling temperature of the organic second solution, but this will require a longer reaction time for polymerization. A particularly suitable azo initiator has an activation temperature that is less than about 53° C. and this azo initiator is used with an organic second solution having a boiling temperature of about 55° C. A particularly suitable azo initiator is VA-044™ (2,2′-azobis(2-[2-imidazolin-2-yl])propane dihydrochloride, commercially available from Wako Pure Chemical Industries, Ltd., Richmond, Va.) having an activation temperature of between 51° C. and 52° C.

The azo initiator has advantages over other initiators such as persulfates and hydroperoxides. The azo initiator is effective when used in very low amounts, in contrast to other initiators. The azo initiator is used at about 0.1% to 1.0% by weight % of monomer. Preferably about 0.5% azo initiator is used. The low level of azo initiator results in very low levels of initiator contamination in the polymerized hydrogel as compared to contamination resulting from use of other initiators. In addition, there is no metal contamination resulting from the azo initiator, while other initiators typically include metal catalysts that do leave metal contamination in the polymerized product. In addition, other typical initiators are sensitive to oxygen, and, therefore, solutions in contact with these initiators must be de-aerated. The remaining oxygen content of the de-aerated solutions is variable, leading to inconsistency in the microsphere forming process. With use of an azo initiator, no de-aeration is required, which reduces the complexity of solution preparation for use in the microsphere formation process and increases the consistency of microsphere preparation. In addition persulfate initiators generally give more inconsistent conversion and yields of microspheres than azo initiators.

Second Solution

An organic solution acts as a dispersion medium in the process of microsphere preparation described by Figuly et al., supra, and is herein called the “second solution”. The second solution comprises at least one substantially chlorinated hydrocarbon of less than 6 carbon units, excluding halogenated aromatic hydrocarbons. A substantially chlorinated hydrocarbon may be a hydrocarbon that is at least 50% chlorinated, as well as a fully chlorinated hydrocarbon. Particularly suitable is a chlorinated solvent that readily dissolves ethyl cellulose to a homogeneous solution, boils above at least about 50° C. and has a density able to support microsphere formation in aqueous/organic suspension. A particularly useful organic medium in the process of microsphere preparation described by Figuly et al., supra is a mixture containing chloroform and methylene chloride. Methylene chloride alone does not have a high enough boiling temperature to allow the use of a low temperature aqueous azo initiator. Chloroform alone is not sufficient to support microsphere formation. The combination of chloroform and methylene chloride provides an organic solution which has a boiling temperature allowing use of a low temperature aqueous azo initiator and which supports microsphere formation in the aqueous/organic suspension. Chloroform and methylene chloride may be used in volume ratios between about 20:1 and about 1:20. More suitable is a chloroform and methylene chloride solution with a volume ratio between about 5:1 and 1:5. Particularly suitable is a volume ratio of 3:1 chloroform:methylene chloride solution which has a boiling temperature of about 53° C.

Additionally, other solvents or solvent mixtures may be used in combination with a substantially chlorinated hydrocarbon such as methylene chloride. For example, it may be desirable to substitute for chloroform in the chloroform-methylene chloride mixtures described above because of the health hazards of chloroform. Suitable solvent or solvent mixtures to substitute for chloroform may be selected by matching the Hansen solubility parameters (Hansen, Hansen Solubility Parameters, A User's Handbook, CRC Press LLC, Boca Raton, Fla., 2000) of particular solvent or solvent mixtures to those of chloroform, as described by Figuly et al., supra. Preferred solvent mixtures have a sum of the differences (in absolute value) in Hansen solubility parameters relative to the Hansen solubility parameters of chloroform of less than about 0.21.

In one embodiment, the second solution comprises a combination of a solvent mixture of 30 vol % (volume percent) ethyl heptanoate (CAS No. 106-30-9) and 70 vol % phenethyl acetate (CAS No. 103-45-7), with methylene chloride in a volume ratio of about 20:1 to about 1:20, in addition about 5:1 to about 1:5, and further in addition of about 3:1.

The second solution also comprises a viscosity modifying component that provides a surface tension that allows droplet formation in the aqueous/organic suspension formed during the microsphere preparation process. This viscosity modifying component is again called a “protecting colloid”. A variety of natural and synthetic compounds soluble in organic media may be used as a protecting colloid, including, but not limited to, cellulose derivatives, polyacrylates (such as polyacrylic acid and polymethacrylic acid), polyalkylene glycols such as polyethylene glycol, partially hydrolyzed polyvinyl alcohol and other polyols, guar gum, and agar gum. Particularly useful are cellulose ethers such as methyl cellulose, hydroxymethyl cellulose, hydroxyethyl cellulose, ethylhydroxyethyl cellulose, hydroxypropyl cellulose, ethyl cellulose, and benzyl cellulose; as well as cellulose esters such as cellulose acetate, cellulose butylate, cellulose acetate butylate, cellulose propionate, cellulose butyrate, cellulose acetate propionate, cellulose acetate butyrate, and cellulose acetate phthalate. The amount of the protecting colloid in the organic second solution is sufficient to reduce microdroplet coalescence in the aqueous/organic suspension, and is generally between about 0.5% and about 5% by weight % of the organic second solution. Particularly suitable is ethyl cellulose at about 1.5% by weight.

Process for Microsphere Preparation

In the process for microsphere preparation described by Figuly et al., supra, the first solution and the second solution are combined with agitation to form a first suspension. The second solution is used in an amount that is adequate to form a good suspension, while the amount may be as great as is practical. Generally the volume ratio of second to first solutions is in the range of about 10:1 to about 2:1. Preferably the volume ratio of second to first solutions is in the range of about 6:1 to about 4:1.

The first and second solutions may be combined in any order. Specifically, the first solution can be added to the second solution, the second solution can be added to the first solution, or the two solutions can be combined simultaneously. Preferably, the first solution is added to the second solution. During the combination of the first and second solutions, the resulting mixture is agitated at a rate capable of forming a uniform suspension from the two solutions. Agitation may be by any method which thoroughly mixes the two solutions, such as shaking or stirring. Typically, the second solution is stirred in a container while the first solution is poured into the same container. The combined first and second solution is agitated at a temperature that is below the azo initiation temperature (and above the freezing point of the solution) to form a uniform, first suspension. Generally the temperature is below about 50° C., and more typically is below about 40° C. A temperature that is below about 30° C. is preferred. Typically the first suspension is stirred at about 100 to 600 rpm, depending on the size of the container, at room temperature for about one-half to one hour.

The agitation of the first suspension allows formation of small droplets in the suspension. The size of the forming droplets, and therefore the size of the microspheres that are produced, is related to the rate of agitation. As the agitation is reduced, droplets coalesce. Agitation is maintained at a rate sufficient to reduce droplet coalescence allowing the formation of micron sized microspheres. For example, for the formation of microspheres in the size range of 40 to 500 microns, stirring is typically about 150-250 rpm when using a one liter container. The optimum agitation rate for any particular system will depend on many factors, including the particular monomer, crosslinking agent, and solvent system used, the geometry of the container, the geometry of the agitator, and the desired microsphere properties for the intended application. For example, the size of the microspheres depends on the agitation rate. In general, larger microspheres are obtained at lower agitation rates. The agitation rate for any given conditions can be readily optimized by one skilled in the art using routine experimentation.

After the formation of the first suspension, a low level of heat is applied such that the temperature of the first suspension is brought to a temperature that is below the boiling temperature of the first solution, and below or at the boiling temperature of the second solution. Typically the temperature is between about 50° C. and 55° C., depending on the mixture of the second solution. It is preferred to bring the temperature of the first suspension made with a chloroform and methylene chloride ratio of about 3:1 to about 51° C. to 52° C. At this temperature the low temperature azo initiator is activated. The first suspension is agitated until it forms a second suspension comprising a precipitate of gelatinous microspheres in the suspending medium, which is predominantly an organic liquid phase. The gelatinous precipitate appears as a milky material which falls out of the suspension. Additionally, a white foam may be seen on top of the second suspension. Typically stirring of the first suspension to form the second suspension at the elevated temperature is for about 8-10 hours. The second suspension is agitated for another period of time at room temperature to ensure that the polymerization and microsphere formation is complete. During this time the second suspension cools to a temperature which is easily handled. Generally this is at or below about 30° C. Room temperature, typically at about 25° C., is conveniently used. Typically stirring remains at about 150-250 rpm, when using a one liter container, for about 8-14 hours.

Agitation is ceased, allowing the formed microspheres to settle to the bottom of the container. Removing the water from these hydrogel microspheres may be accomplished by washing with a dehydrating solvent such as methanol, ethanol, or acetone. Particularly useful is methanol, which is added, and the mixture is optionally agitated gently for about an hour to allow good solvent exchange. The microspheres are then recovered by a method such as by decanting or filtering, and may be washed a second time with methanol and again recovered. With removal of the water, the microspheres change in appearance from milky and gelatinous to hard and opaque white. The microspheres finally may be washed in ethanol, which is desirable for removal of residual methanol, particularly for microsphere use in medical applications. The washed microspheres in ethanol form one type of microsphere slurry. The microspheres optionally may be dried to form a powder of microspheres. Drying rids the microspheres of remaining washing solvent and additional water. Drying may be by any standard method such as using air, heat, and/or vacuum. Particularly useful is drying under vacuum in a vacuum oven set at about 20° C. to about 100° C. with a nitrogen purge. The use of lower drying temperatures requires longer drying times. For preparation of highly swellable microspheres, drying at room temperature (i.e., about 20° C. to about 25° C.) under vacuum with a nitrogen purge is preferred (see Example 34). A small amount of water generally remains in the microspheres after drying. The amount of remaining water may be about 1% to 10% of the microsphere total weight. The resulting microsphere preparation, though retaining a small amount of water in the microspheres, flows when tilted or swirled in a container and thus forms a free-flowing microsphere powder.

The microspheres prepared by the method of Figuly et al., supra have properties of general consistency in size and shape, high density, low fracture, an interior closed-cell voided structure, high swell capacity, rapid swell, and deformability following swell.

### Water-Swellable Hydrogel Microspheres Comprising an Active Agent

For applications which involve delivery of an active agent, such as a pharmaceutical drug, therapeutic agent, fertilizer, herbicide, or pesticide, microspheres prepared by methods known in the art may be prepared to comprise the desired active agent. The active agent may be loaded into the microspheres using various methods known in the art. For example, the microspheres may be imbibed with the agent by swelling the microspheres in a medium containing the agent and allowing it to soak into the microspheres. The microspheres may then be dried or deswelled by removing water by washing with a dehydrating solvent, as described above. Additionally, the active agent may be coated onto the microspheres using methods such as spraying, immersion, and the like. The active agent may also be directly incorporated into the microspheres during their preparation.

In one embodiment, the water-swellable hydrogel microspheres comprise a pharmaceutical drug or therapeutic agent. Suitable pharmaceutical drugs and therapeutic agents are well known in the art. An extensive list is given by Kabonov et al. in U.S. Pat. No. 6,696,089 (in particular, columns 16 to 18). Examples include, but are not limited to, antibacterial agents, antiviral agents, antifungal agents, anti-cancer agents, vaccines, anti-inflammatories, anti-glaucomic agents, analgesics, local anesthetics, anti-neoplastic agents, anti-angiogenic agents, and the like.

### Method of Making Dimensionally Stable, Shaped Articles

In the method of making dimensionally stable, shaped, articles comprised of dried, aggregated, water-swellable hydrogel microspheres disclosed herein, a suspension of water-swellable hydrogel microspheres in an aqueous medium is provided in a mold having the desired preselected shape. The preselected shape may be any suitable shape depending on the intended application of the article. Suitable shapes include, but are not limited to, a cylinder, a rod, a disk, a disk with a center hole, a star, a flower, a cube, a spike, and the like. The aqueous medium may be water or a mixture of water and a volatile, water-miscible organic solvent such as aliphatic alcohols, polyhydric alcohols, amides, ketones, and the like. In one embodiment, the aqueous medium is water. If a mixture of water and a volatile, water-miscible organic solvent is used as the aqueous medium, the amount of water present in the mixture is adjusted so that the microspheres are at least partially swollen. By “partially swollen” is meant that the microspheres have absorbed some amount of water below their swell capacity, and have expanded in size. Preferably, the microspheres are swollen to at least 25% of their swell capacity. In one embodiment, the microspheres are swollen to 100% of their swell capacity. For microspheres having a very high swell capacity and therefore a very high water content when fully swollen, i.e., microspheres comprised of sulfonic acid or a combination of sulfonic acid and the sodium salt of sulfonic acid, shapes having higher dimensional stability may be obtained using partially swollen microspheres rather than microspheres swollen to 100% of their swell capacity. The swell capacity of the microspheres can be measured by determining the maximum weight of water absorbed per weight of microspheres using the method described in Example 1 herein below, wherein the amount of water used is sufficient to fully swell the microspheres.

Next, the suspension is evaporatively dried to remove substantially all of the aqueous medium to form the dimensionally stable, shaped article. A small amount of aqueous medium may remain in the microspheres after drying. The amount of remaining water may be about 1% to about 10% of the total weight of the microspheres. The evaporative drying is done to slowly remove the aqueous medium from the microspheres. The evaporative drying may be done passively at ambient conditions of temperature and humidity, for example a temperature of about 18° C. to about 25° C. and a relative humidity of about 30% to about 50%. A gentle flow of a gas such as air or nitrogen over the suspension at ambient temperature may also be used for the evaporative drying in some cases, depending on the composition of the microspheres used, as described below. Additionally, the removal of the aqueous medium may be done under controlled conditions of temperature and humidity at which the rate of water removal is comparable to the rate at ambient conditions. For example, the suspension may be evaporatively dried in a controlled humidity chamber at a temperature above ambient temperature, while keeping the humidity constant at a value above ambient humidity to slow the rate of drying. Preferably, the suspension is evaporatively dried passively at ambient conditions of temperature and humidity.

The composition of the microspheres plays a roll in determining the drying conditions necessary to form a dimensionally stable article, as shown in Example 5 herein. Microspheres comprised of monomers having a low glass transition temperature (i.e., 95% acrylic acid and 5% 2-hydroxylethyl acrylate) were less sensitive to drying conditions, specifically, they could be dried using a gentle flow of gas at ambient temperature. However, microspheres comprised of 95% acrylic acid and 5% 2-hydroxylethyl methacrylate gave more stable shapes when dried passively at ambient conditions of temperature and humidity than when dried using a gentle flow of gas. The lower glass transition temperature of hydroxylethyl acrylate as compared to that of hydroxylethyl methacrylate could give rise to less brittle structures and thus aide in the generation of more integrated, dimensionally stable shapes.

Upon drying, the microspheres aggregate together forming the shaped article, which retains the preselected shape of the mold, but not necessarily the size of the mold. Typically, the size of the shaped article is smaller than the size of the mold. The size of the shaped article may be controlled by varying the amount of water-swellable hydrogel microspheres in the starting suspension of microspheres in the aqueous medium.

The method of making the shaped article is carried out in the absence of a binding agent, such as a binder, glue, an external crosslinking agent, or the like, that would bind the microspheres together; therefore, the resulting shaped article does not comprise a binding agent to bind the microspheres together. The absence of any binding agent allows the shaped article to swell and disaggregate when placed in an aqueous medium in a second container, thereby forming at least partially swollen hydrogel microspheres, which take the shape of the second container. The second container may be the same as or different from the original container holding the starting suspension of water-swellable hydrogel microspheres.

### Applications of the Dimensionally Stable, Shaped Articles

The dimensionally stable, shaped articles can be made in a variety of shapes and may be used for various applications, including medical applications such as a medical implant for tissue augmentation, void filling, wound treatment, spinal disk reconstruction, joint repair, embolization, drug delivery, as a plug for the punctum to treat dry eye syndrome, as a plug to treat urinary incontinence, and as plug to seal a fistula. Additionally, the dimensionally stable, shaped articles disclosed herein may be useful for other applications including, but not limited to, absorption applications, such as small-scale spill control; and delivery applications to carry and release active ingredients such as a spike that may be inserted into the ground to release fertilizers, herbicides, and pesticides.

In one embodiment, the dimensionally stable, shaped article comprising dried, water-swellable hydrogel microspheres is a medical implant.

In another embodiment, the invention provides a method for completely or partially blocking or filling a lumen or void within the body of a mammal. In the method, a dimensionally stable, shaped article comprised of dried, water-swellable hydrogel microspheres, aggregated to form a predetermined shape, as described above, is implanted into a lumen or void within the body. The dimensionally stable, shaped article is then allowed to swell and disaggregate upon exposure to a physiological aqueous fluid present in or surrounding the lumen or void, thereby forming at least partially swollen hydrogel microspheres, which totally or partially block or fill the lumen or void. The shaped article may be implanted through a surgical incision or an open lumen, such as the punctum to treat dry eye syndrome or a fistula. Although useful for a variety of medical applications, as noted above, the dimensionally stable, shaped article disclosed herein may be particularly advantageous for filling voids created by congenital abnormalities, disease, aging, damage due to trauma (e.g., facial reconstruction) and/or surgery such as extraction of tumors and other growth masses; spinal disk reconstruction; joint repair; blocking the punctum to treat dry eye syndrome; and sealing a fistula.

### EXAMPLES

The present invention is further defined in the following Examples. These Examples are given by way of illustration only, and should not be construed as limiting. From the above discussion and these Examples, one skilled in the art can ascertain the essential characteristics of this invention, and without departing from the spirit and scope thereof, can make various changes and modifications of the invention to adapt it to various uses and conditions.

### General Materials

Chemicals, solvents, and other ingredients were purchased from Aldrich (Milwaukee, Wis.) and used as received, unless otherwise specified. The VA-044 polymerization initiator was used as received from Wako Pure Chemical Industries, Ltd (Richmond, Va.).

### Example 1

### Preparation of a Disk-Shaped Pellet Comprised of Dried, Aggregated, Water-Swellable Hydrogel Microspheres

The purpose of this Example was to demonstrate the preparation of a disk-shaped pellet comprised of dried, aggregated, water-swellable hydrogel microspheres prepared from acrylic acid and 2-hydroxylethyl methacrylate.

Hydrogel microspheres containing 95% acrylic acid and 5% 2-hydroxylethyl methacrylate were prepared according to the method described by Figuly et al. in U.S. Patent Application Publication No. 2007/0237956, Example 28.

In a 150 mL preweighed, coarse fritted funnel, was added 0.503 g of the dried hydrogel microspheres. The stem of the funnel was sealed with a rubber stopper. Then, the funnel was placed on a filter flask and 150 mL of distilled water, an amount sufficient to completely swell the microspheres, was added to the funnel and its contents at room temperature. The contents were left undisturbed for 15 min. The stopper was then removed from the stem of the funnel, and suction was applied for 5 min. The stem and the underside of the funnel were then rinsed with ethanol to remove any remaining water droplets and suction was continued for an additional 5 min. Any remaining water droplets were wiped off the funnel. The funnel and contents were weighed to determine the weight of water retained by the microspheres. The swell capacity was calculated to be 111 g of water/g of dried microsphere as follows:

$$\begin{matrix}swell = \frac{[ \begin{matrix}( total mass of wet microspheres + funnel ) - \\ ( total mass of dry microspheres + funnel )\end{matrix} ] }{mass of dry microspheres} \\ = \frac{[ wet mass of microspheres - dry mass of microspheres ]}{dry mass of microspheres} \\ = \frac{mass water retained ( g )}{mass of dry microspheres ( g )}\end{matrix}$$

After weighing, the microspheres were evaporatively dried at ambient conditions of temperature and humidity in the fritted funnel, which served as the mold for the disc-shaped pellet. After approximately two weeks of drying, the individual microspheres had concentrated in the form of a rigid, dimensionally stable, disc-shaped pellet. The internal diameter of the fritted funnel was 6.7 cm and the disc-shaped pellet diameter was approximately 1 cm.

FIGS. 1a and 1 b show electron micrographs of the aggregated microsphere disc-shaped pellet at low and high magnification, respectively. As can be seen from the figures, the surfaces of the microspheres are integrated to some degree, resulting in a dimensionally stable pellet that can be handled without breaking apart.

### Example 2

### Preparation of a Disk-Shaped Pellet Comprised of Dried, Aggregated, Water-Swellable Hydrogel Microspheres

The purpose of this Example was to demonstrate the preparation of a disk-shaped pellet comprised of dried, aggregated, water-swellable hydrogel microspheres prepared from acrylic acid and 2-hydroxylethyl acrylate.

Hydrogel microspheres containing 95% acrylic acid and 5% 2-hydroxylethyl acrylate were prepared according to the method described by Figuly et al., supra, Example 31.

The dried hydrogel microspheres were suspended in water, weighed to determine swell, and then evaporatively dried as described in Example 1 to give an aggregated microsphere, disc-shaped pellet. FIGS. 2a and 2b show electron micrographs of the aggregated microsphere pellet at low and high magnification, respectively. As can be seen from the figures, the surfaces of the microspheres are integrated to some degree, resulting in a dimensionally stable pellet that can be handled without breaking apart.

### Example 3

### Preparation of a Disk-Shaped Pellet Comprised of Dried, Aggregated, Water-Swellable Hydrogel Microspheres

The purpose of this Example was to demonstrate the preparation of a disk-shaped pellet comprised of dried, aggregated, water-swellable hydrogel microspheres prepared from acrylic acid.

Hydrogel microspheres containing 100% acrylic acid were prepared according to the method described by Figuly et al., supra, Example 1.

The dried hydrogel microspheres were suspended in water, weighed to determine swell, and then evaporatively dried as described in Example 1 to give an aggregated microsphere disc-shaped pellet.

### Example 4

### Effect of Drying Conditions on Shaped Article Formation

The purpose of this Example was to demonstrate the effect of drying conditions on the formation of a shaped article comprised of dried, aggregated, water-swellable hydrogel microspheres prepared from acrylic acid and 2-hydroxylethyl methacrylate.

Hydrogel microspheres containing 95% acrylic acid and 5% 2-hydroxylethyl methacrylate were prepared according to the method described by Figuly et al., supra, Example 28.

To each of three flower-shaped silicone baking molds, was added 0.5 g of the dried hydrogel microspheres. To each of these molds, 92.2 g of water was added, the amount of which was enough to completely swell the microspheres. After all the microspheres were swollen, each mold was dried using a different drying condition: (1) ambient conditions (slowest drying condition), (2) inside a chemical fume hood with air flow (intermediate drying condition), and (3) in a vacuum oven at room temperature under a nitrogen purge without vacuum (fastest drying condition). After 10 days, the shaped articles were examined.

An intact flower-shaped article comprised of the aggregated microspheres was obtained with drying at ambient conditions for approximately 50 days. At the faster drying conditions, the flower shapes fell apart within 10 days. These results demonstrate the need for slow drying conditions to form a stable shaped article.

### Example 5

### Effect of Drying Conditions on Shaped Article Formation

The purpose of this Example was to demonstrate the effect of drying conditions on the formation of a shaped article comprised of dried, aggregated, water-swellable hydrogel microspheres prepared from acrylic acid and 2-hydroxylethyl acrylate.

Hydrogel microspheres containing 95% acrylic acid and 5% 2-hydroxylethyl acrylate were prepared according to the method described by Figuly et al., supra, Example 31.

To each of three star-shaped silicone baking molds, was added 0.4 g of the dried hydrogel microspheres. To each of these molds, 59.6 g of water was added to completely swell the microspheres. After all the microspheres were swollen, each mold was dried using a different drying condition: (1) ambient conditions (slowest drying condition), (2) inside a chemical fume hood with air flow (intermediate drying condition), and (3) in a vacuum oven at room temperature under a nitrogen purge without vacuum (fastest drying condition).

An intact star-shaped article comprised of the aggregated microspheres was obtained with all three drying conditions. With drying conditions (2) and (3), the faster drying conditions, the star-shaped articles were formed within 10 days. It took close to 50 days to form the star-shaped article with ambient drying conditions. Minor surface cracks were evident in the star-shaped article formed by drying in the oven under nitrogen purge (the fastest drying condition). These results demonstrate that the composition of the microsphere plays a roll in determining the drying conditions necessary to form an intact article. Microspheres comprised of 95% acrylic acid and 5% 2-hydroxylethyl acrylate were less sensitive to drying conditions than the microspheres comprised of 95% acrylic acid and 5% 2-hydroxylethyl methacrylate described in Example 4. The lower glass transition temperature of hydroxylethyl acrylate as compared to that of hydroxylethyl methacrylate could give rise to less brittle structures and thus aide in the generation of more integrated dimensionally stable shapes.

### Examples 6-14

### Preparation of Various Shaped Articles Comprised of Dried, Aggregated, Water-Swellable Hydrogel Microspheres

The purpose of these Examples was to demonstrate that various shaped articles comprised of dried, aggregated, water-swellable hydrogel microspheres can be prepared using the method disclosed herein.

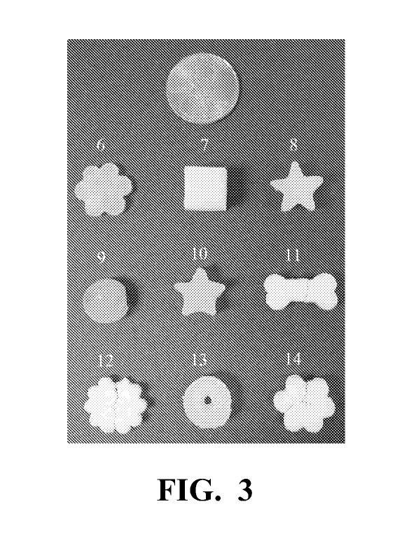

The microsphere compositions, mold shapes used, and evaporative drying conditions are summarized in Table 1. The resulting shaped articles are shown in FIG. 3. For size comparison, a United States penny is included in the figure. These results demonstrate that shaped articles comprised of dried, aggregated, water-swellable microspheres may be formed in virtually any shape.

TABLE 1
Summary of Conditions for Preparation of Various Shaped Articles

| | Microsphere | | |
|---|---|---|---|
| Example | Composition | Mold Shape | Drying Conditions |
| 6 | 95% AA/5% HEA¹ | flower | combination of |
| | | | ambient conditions |
| | | | and drying in a |
| | | | fume hood with air |
| | | | flow |
| 7 | 60% AA/40% HEA² | cube | combination of |
| | | | ambient conditions |
| | | | and drying in a |
| | | | fume hood with air |
| | | | flow |
| 8 | 95% AA/5% HEA¹ | star | drying in fume |
| | | | hood with air flow |
| 9 | 95% AA/5% HEA¹ | cylinder | drying in fume |
| | | | hood with air flow |
| 10 | 95% AA/5% HEA¹ | star | ambient conditions |
| 11 | 95% AA/5% HEMA³ | dog bone | ambient conditions |
| 12 | 95% AA/5% HEMA³ | flower | ambient conditions |
| 13 | 95% AA/5% HEA¹ | cylinder⁴ | combination of |
| | | | ambient conditions |
| | | | and drying in a |
| | | | fume hood with air |
| | | | flow |
| 14 | 95% AA/5% HEMA³ | flower | ambient conditions |

¹Prepared according to Example 31 in Figuly et al., supra.
²Prepared according to Example 33 in Figuly et al., supra.
³Prepared according to Example 28 in Figuly et al., supra.
⁴After partial drying, the center of the microsphere disc was removed with a cork borer to obtain an annular shape.

### Example 15

### Shape Transformation of an Article Formed from Dried, Aggregated, Water-Swellable Microspheres

The purpose of this Example was to demonstrate that a shaped article formed from dried, aggregated, water-swellable microspheres according to the method disclosed herein, undergoes a shape transformation upon immersing the article in a water-containing mold having a different shape.

The star-shaped article described in Example 5, prepared with drying in a vacuum oven at room temperature with a nitrogen purge, was immersed in a flower-shaped silicone mold filled with 80 g of water. The individual microspheres hydrated slowly and separated from the star-shaped article, forming swollen microspheres that conformed to the shape of the new mold, i.e., the flower. It took 3 h and 44 min for the microspheres to completely hydrate and take the shape of the new mold. The swollen microspheres were then dried in the flower-shaped mold using a combination of drying at ambient conditions and drying in a chemical fume hood with air flow. As the water started to evaporate over a period of time, the microspheres came together and aggregated in the form of the new shape i.e., the flower, with no memory of the previous shape. The microspheres eventually aggregated together in the shape of a flower-shaped pellet. The complete transformation from dry star shape to dry flower shape took a total period of 4 weeks. This result demonstrates that a shaped article comprised of dried, aggregated, water-swellable microspheres, prepared according to the method disclosed herein, will loose its shape when hydrated, and take the shape of the new container.

### Example 16

### Preparation of a Disk-Shaped Pellet Comprised of Dried, Aggregated, Water-Swellable Hydrogel Microspheres

The purpose of this Example was to demonstrate the preparation of a disk-shaped pellet comprised of dried, aggregated, water-swellable hydrogel microspheres prepared from styrene sulfonic acid.

Hydrogel microspheres containing styrene sulfonic acid were prepared according to the method described by Figuly et al. in U.S. Patent Application Publication No. 2007/0237956, Example 36. A typical preparation is described below.

In a 1 L round-bottom, three-necked flask equipped with an overhead stirrer, thermometer, reflux condenser, and nitrogen inlet port is prepared a solution of 6.0 g of ethyl cellulose, 269 mL of chloroform, and 97 g of methylene chloride (solution A). The mixture is stirred at 244 rpm until the ethyl cellulose dissolved. In a second flask, is prepared a solution of 0.25 g methyl cellulose, 0.175 g of N,N′-methylenebisacrylamide (2.4 Mol % of monomer), 4.335 g Triton™ X-405 (polyoxyethylene (40) isooctylphenyl ether—70% solution in water), and 5.0 g of water (solution B). In a third separate flask is mixed 9.75 g of 4-styrenesulfonic acid, sodium salt hydrate (0.048 mol) and 17.24 g of a 10% HCl solution (0.048 mol; to convert the sodium salt of the monomer to the acid form), also 28.9 g of water is added to this solution (to reach a pH of 0) (solution C). The monomer solution is then added to the crosslinker solution (solution B). The total amount of water in the medium is 49.4 g, including that from the HCl.

At this point while rapidly stirring the mixture of solutions B and C, 0.025 g of the water-soluble azo initiator VA-044 (2,2′-azobis(2-[2-imidazolin2-yl])propane dihydrochloride) is added, and the resulting solution is stirred for 5 min. This solution (the “first solution”) is then added to the round-bottom flask containing solution A (the “second solution”). The resulting reaction mixture is allowed to stir (the “first suspension”) at 235 rpm for about 1 h at room temperature. The stirring speed is reduced to 224 rpm and the first suspension is heated to 50.3° C. The suspension is maintained at the same stirring rate and temperature for almost 6 hours to allow for substantial microsphere formation (the “second suspension”). The second suspension is then stirred at 223 rpm for another 14 hours at room temperature to ensure complete polymerization. After this time, approximately 250 mL of methanol is slowly added to the second suspension to remove water from the microspheres, and the microspheres are stirred an additional hour. The microspheres are then filtered, resulting in a soft mass, which is washed with acetone and then filtered again. The material is further washed with 100 mL of methanol and washed again twice with 80 mL portions of ethanol. Finally the solids are dried in a nitrogen purged vacuum oven set at 100° C. The resulting microspheres are obtained as a fine powder with a yellow tint.

To a round silicone baking mold having a diameter of 2 inches (5.1 cm), 0.5 g of sulfonic acid microspheres was added. Then, 25 mL of water was added to wet the microspheres. The wetted microspheres were left on an open bench top to dry at ambient conditions over a period of a few days. After 6 days a yellow disc-shaped pellet approximately 0.03 inches (0.8 mm) thick and 4 cm in diameter, having curled up edges, was formed. The appearance of the disc-shaped pellet was different from the disk shaped pellet formed from acrylic acid and 2-hydroxylethyl methacrylate microspheres (Example 1) in that the microspheres were almost totally fused together forming an amorphous structure in which individual microspheres were not discernable.

### Example 17

### Preparation of a Disk-Shaped Pellet Comprised of Dried, Aggregated, Water-Swellable Hydrogel Microspheres

The purpose of this Example was to demonstrate the preparation of a star-shaped pellet comprised of dried, aggregated, water-swellable hydrogel microspheres prepared from styrene sulfonic acid.

To a star-shaped silicone baking mold, 0.5 g of sulfonic acid microspheres (prepared as described in Example 16) was added. Then, 12.5 g of water was added to wet the microspheres. The microspheres and water were mixed gently using a spatula. Some air bubbles were observed to be trapped as the microspheres started to absorb water and transform into a gel. The wetted microspheres were left on an open bench top at ambient conditions to dry over a period of a few days. After a few days a star-shaped pellet was formed, which had visible bubbles embedded in it. The star-shaped pellet had the same appearance as the disk-shaped pellet described in Example 16.

### Example 18

### Shape Transformation of an Article Formed from Dried, Aggregated, Water-Swellable Microspheres

The purpose of this Example was to demonstrate that a shaped article formed from dried, aggregated, water-swellable styrene sulfonic acid microspheres, prepared according to the method disclosed herein, undergoes a shape transformation upon immersing the article in a water-containing mold having a different shape.

The star-shaped pellet described in Example 17 (weighing 0.48 g) was placed in a flower-shaped silicone mold containing 20 g of water. Within approximately 34 min, the star-shape of the pellet was lost and the solid pellet was transformed into a gel-like state taking the shape of the new flower-shaped silicone mold. The mold was left on an open bench top at ambient conditions to dry over a period of a few days. After 5 days, a flower-shaped pellet was obtained which was completely detached from the new mold. This result demonstrates that a shaped article comprised of dried, aggregated, water-swellable microspheres, prepared according to the method disclosed herein, will loose its shape when hydrated, and take the shape of the new container.

1. A dimensionally stable, shaped article comprising dried, water-swellable hydrogel microspheres, aggregated to form a predetermined shape, wherein said article does not contain a binding agent to bind the microspheres together.

2. The dimensionally stable, shaped article according to claim 1, wherein the water-swellable hydrogel microspheres comprise at least one monomer selected from the group consisting of acrylic acid, methacrylic acid, salts of acrylic acid and methacrylic acid, acrylamide, methacrylamide, N-substituted acrylamides, N-substituted methacrylamides, vinyl alcohol, vinyl acetate, methyl maleate, 2-acryloylethane-sulfonic acid, 2-methacryloylethane-sulfonic acid, salts of 2-acryloylethane-sulfonic acid and 2-methacryloylethane-sulfonic acid, styrene-sulfonic acid, salts of styrene-sulfonic acid, 2-hydroxyethyl acrylate, 2-hydroxyethyl methacrylate, isobutylene, maleic anhydride, acrylonitrile, and ethylene glycol.

3. The dimensionally stable, shaped article according to claim 2, wherein the water-swellable hydrogel microspheres comprise acrylic acid and at least one monomer selected from the group consisting of sodium acrylate, 2-hydroxyethyl methacrylate, 2-hydroxyethyl acrylate, styrene sulfonic acid, and the sodium salt of styrene sulfonic acid.

4. The dimensionally stable, shaped article according to claim 2, wherein the water-swellable hydrogel microspheres comprise styrene sulfonic acid or a combination comprising styrene sulfonic acid and the sodium salt of styrene sulfonic acid.

5. The dimensionally stable, shaped article according to claim 2, wherein the water-swellable hydrogel microspheres comprise acrylic acid, sodium acrylate and vinyl alcohol.

6. The dimensionally stable, shaped article according to claim 1, wherein said shaped article is a medical implant.

7. (canceled)

8. The dimensionally stable, shaped article according to claim 1, wherein the water-swellable hydrogel microspheres are made by a process comprising the steps of:
a) forming a first solution comprising:
(i) water;
(ii) at least one water miscible monomer selected from the group consisting of acrylic acid, methacrylic acid, salts of acrylic acid and methacylic acid, acrylamide, methacrylamide, N-substituted acrylamides, N-substituted methacrylamides, 2-acryloylethane-sulfonic acid, 2-methacryloylethane-sulfonic acid, salts of 2-acryloylethane-sulfonic acid and 2-methacryloylethane-sulfonic acid, styrene-sulfonic acid, salts of styrene-sulfonic acid, 2-hydroxyethyl acrylate, and 2-hydroxyethyl methacrylate; provided that:
(A) if said at least one water miscible monomer is acrylamide, methacrylamide, N-substituted acrylamides, 2-hydroxyethyl acrylate, or 2-hydroxyethyl methacrylate, said monomer is used in combination with at least one other monomer selected from subgroup 1 consisting of: acrylic acid, methacrylic acid, salts of acrylic acid and methacylic acid, 2-acryloylethane-sulfonic acid, 2-methacryloylethane-sulfonic acid, salts of 2-acryloylethane-sulfonic acid and 2-methacryloylethane-sulfonic acid, styrene-sulfonic acid, and salts of styrene-sulfonic acid;
(B) if said first solution contains at least one water miscible monomer from subgroup 2 consisting of acrylic acid, methacrylic acid, salts of acrylic acid and methacylic acid, acrylamide, methacrylamide, N-substituted acrylamides, N-substituted methacrylamides, 2-hydroxyethyl acrylate, and 2-hydroxyethyl methacrylate, but does not contain a monomer selected from subgroup 3 consisting of 2-acryloylethane-sulfonic acid, 2-methacryloylethane-sulfonic acid, salts of 2-acryloylethane-sulfonic acid and 2-methacryloylethane-sulfonic acid, styrene-sulfonic acid, and salts of styrene-sulfonic acid, then the pH of the first solution is at least about 3;
(C) if said first solution contains at least one water miscible monomer from subgroup 3 consisting of 2-acryloylethane-sulfonic acid, 2-methacryloylethane-sulfonic acid, salts of 2-acryloylethane-sulfonic acid and 2-methacryloylethane-sulfonic acid, styrene-sulfonic acid, and salts of styrene-sulfonic acid, then the pH of the first solution is less than about 3;
(iii) a crosslinking agent that is miscible in the first solution in less than or equal to about 5 Mol %, relative to total moles of monomer and crosslinking agent, said crosslinking agent being selected from the group consisting of N,N′-methylene-bis-acrylamide, N,N′-methylene-bis-methacrylamide, N-methylolacrylamide, N-methylolmethacrylamide, glycidyl acrylate, glycidyl methacrylate, polyethylene glycol diacrylate, polyethylene glycol dimethacrylate, polyvalent metal salts of acrylic acid and methacrylic acid, divinyl benzene phosphoacrylates, divinylbenzene, divinylphenylphosphine, divinyl sulfone, 1,3-divinyltetramethyldisiloxane, 3,9-divinyl-2,4,8,10-tetraoxaspiro[5,5]undecane, phosphomethacrylates, ethylene glycol diglycidyl ether, glycerin triglycidyl ether, glycerin diglycidyl ether, and polyethylene glycol diglycidyl ether;
(iv) a water soluble protecting colloid;
(v) an emulsifier; and
(vi) a low temperature aqueous soluble azo initiator;
b) forming a second solution comprising at least one substantially chlorinated hydrocarbon of less than 6 carbon units, provided that the chlorinated hydrocarbon is not a halogenated aromatic hydrocarbon, and an organic soluble protecting colloid;
c) forming a first suspension with agitation comprising the first and second solutions at a temperature below the initiation temperature of the azo initiator of (a);
d) increasing the temperature of the agitating first suspension to a temperature at which the low temperature aqueous soluble azo initiator is activated;
e) agitating the first suspension until it forms a second suspension comprising a gelatinous precipitate suspended in an organic liquid phase, wherein microspheres are formed;
f) allowing the second suspension to cool to a temperature that is at or below about 30° C. while agitating the second suspension;
g) washing the second suspension at least once with a dehydrating solvent wherein water is removed from the microspheres forming a microsphere preparation; and
h) recovering the microsphere preparation.

9. The dimensionally stable, shaped article according to claim 8, wherein the second solution comprises a mixture of methylene chloride and chloroform.

10. The dimensionally stable, shaped article according to claim 8, wherein the azo initiator is 2,2′-azobis(2-[2-imidazolin-2-yl])propane dihydrochloride.

11. The dimensionally stable, shaped article according to claim 8, wherein the crosslinking agent is N,N′-methylenebisacrylamide.

12. (canceled)

13. A method of making a dimensionally stable, shaped article comprised of dried, aggregated, water-swellable hydrogel microspheres comprising the steps of:
a) providing in a mold having a preselected shape, a suspension of water-swellable hydrogel microspheres in an aqueous medium wherein said microspheres are at least partially swollen; and
b) evaporatively drying said suspension to remove substantially all of the aqueous medium to form the dimensionally stable, shaped article;
wherein:
(i) said method is carried out in the absence of a binding agent; and
(ii) said dimensionally stable shaped article has the shape of the mold.

14. The method according to claim 13, wherein the water-swellable hydrogel microspheres comprise at least one monomer selected from the group consisting of acrylic acid, methacrylic acid, salts of acrylic acid and methacrylic acid, acrylamide, methacrylamide, N-substituted acrylamides, N-substituted methacrylamides, vinyl alcohol, vinyl acetate, methyl maleate, 2-acryloylethane-sulfonic acid, 2-methacryloylethane-sulfonic acid, salts of 2-acryloylethane-sulfonic acid and 2-methacryloylethane-sulfonic acid, styrene-sulfonic acid, salts of styrene-sulfonic acid, 2-hydroxyethyl acrylate, 2-hydroxyethyl methacrylate, isobutylene, maleic anhydride, acrylonitrile, and ethylene glycol.

15. The method according to claim 14, wherein the water-swellable hydrogel microspheres comprise acrylic acid and at least one monomer selected from the group consisting of sodium acrylate, 2-hydroxyethyl methacrylate, 2-hydroxyethyl acrylate, styrene sulfonic acid, and sulfonic acid sodium salt.

16. The method according to claim 14, wherein the water-swellable hydrogel microspheres comprise styrene sulfonic acid or a combination comprising styrene sulfonic acid and styrene sulfonic acid sodium salt.

17. The method according to claim 14, wherein the water-swellable hydrogel microspheres comprise acrylic acid, sodium acrylate and vinyl alcohol.

18. The method according to claim 13, wherein the dimensionally stable, shaped article is a medical implant.

19. The method according to claim 13, wherein the aqueous medium is water.

20. The method according to claim 13, wherein the evaporatively drying is done passively at ambient conditions of temperature and humidity.

21. The method according to claim 13, wherein the water-swellable hydrogel microspheres are made by a process comprising the steps of:
a) forming a first solution comprising:
(i) water;
(ii) at least one water miscible monomer selected from the group consisting of acrylic acid, methacrylic acid, salts of acrylic acid and methacylic acid, acrylamide, methacrylamide, N-substituted acrylamides, N-substituted methacrylamides, 2-acryloylethane-sulfonic acid, 2-methacryloylethane-sulfonic acid, salts of 2-acryloylethane-sulfonic acid and 2-methacryloylethane-sulfonic acid, styrene-sulfonic acid, salts of styrene-sulfonic acid, 2-hydroxyethyl acrylate, and 2-hydroxyethyl methacrylate; provided that:
(A) if said at least one water miscible monomer is acrylamide, methacrylamide, N-substituted acrylamides, 2-hydroxyethyl acrylate, or 2-hydroxyethyl methacrylate, said monomer is used in combination with at least one other monomer selected from subgroup 1 consisting of: acrylic acid, methacrylic acid, salts of acrylic acid and methacylic acid, 2-acryloylethane-sulfonic acid, 2-methacryloylethane-sulfonic acid, salts of 2-acryloylethane-sulfonic acid and 2-methacryloylethane-sulfonic acid, styrene-sulfonic acid, and salts of styrene-sulfonic acid;
(B) if said first solution contains at least one water miscible monomer from subgroup 2 consisting of acrylic acid, methacrylic acid, salts of acrylic acid and methacylic acid, acrylamide, methacrylamide, N-substituted acrylamides, N-substituted methacrylamides, 2-hydroxyethyl acrylate, and 2-hydroxyethyl methacrylate, but does not contain a monomer selected from subgroup 3 consisting of 2-acryloylethane-sulfonic acid, 2-methacryloylethane-sulfonic acid, salts of 2-acryloylethane-sulfonic acid and 2-methacryloylethane-sulfonic acid, styrene-sulfonic acid, and salts of styrene-sulfonic acid, then the pH of the first solution is at least about 3;
(C) if said first solution contains at least one water miscible monomer from subgroup 3 consisting of 2-acryloylethane-sulfonic acid, 2-methacryloylethane-sulfonic acid, salts of 2-acryloylethane-sulfonic acid and 2-methacryloylethane-sulfonic acid, styrene-sulfonic acid, and salts of styrene-sulfonic acid, then the pH of the first solution is less than about 3;
(iii) a crosslinking agent that is miscible in the first solution in less than or equal to about 5 Mol %, relative to total moles of monomer and crosslinking agent, said crosslinking agent being selected from the group consisting of N,N′-methylene-bis-acrylamide, N,N′-methylene-bis-methacrylamide, N-methylolacrylamide, N-methylolmethacrylamide, glycidyl acrylate, glycidyl methacrylate, polyethylene glycol diacrylate, polyethylene glycol dimethacrylate, polyvalent metal salts of acrylic acid and methacrylic acid, divinyl benzene phosphoacrylates, divinylbenzene, divinylphenylphosphine, divinyl sulfone, 1,3-divinyltetramethyldisiloxane, 3,9-divinyl-2,4,8,10-tetraoxaspiro[5,5]undecane, phosphomethacrylates, ethylene glycol diglycidyl ether, glycerin triglycidyl ether, glycerin diglycidyl ether, and polyethylene glycol diglycidyl ether;
(iv) a water soluble protecting colloid;
(v) an emulsifier; and
(vi) a low temperature aqueous soluble azo initiator;
b) forming a second solution comprising at least one substantially chlorinated hydrocarbon of less than 6 carbon units, provided that the chlorinated hydrocarbon is not a halogenated aromatic hydrocarbon, and an organic soluble protecting colloid;
c) forming a first suspension with agitation comprising the first and second solutions at a temperature below the initiation temperature of the azo initiator of (a);
d) increasing the temperature of the agitating first suspension to a temperature at which the low temperature aqueous soluble azo initiator is activated;
e) agitating the first suspension until it forms a second suspension comprising a gelatinous precipitate suspended in an organic liquid phase, wherein microspheres are formed;
f) allowing the second suspension to cool to a temperature that is at or below about 30° C. while agitating the second suspension;
g) washing the second suspension at least once with a dehydrating solvent wherein water is removed from the microspheres forming a microsphere preparation; and
h) recovering the microsphere preparation.

22. (canceled)

23. (canceled)

24. (canceled)

25. A method for completely or partially blocking or filling a lumen or void within the body of a mammal comprising the steps of:
a) providing a dimensionally stable, shaped article comprised of dried, water-swellable hydrogel microspheres, aggregated to form a predetermined shape, wherein said article does not contain a binding agent to bind the microspheres together;
b) implanting the dimensionally stable, shaped article into a lumen or void within the body; and
c) allowing the dimensionally stable, shaped article to swell and disaggregate upon exposure to a physiological aqueous fluid present in or surrounding the lumen or void, thereby forming at least partially swollen hydrogel microspheres, which totally or partially block or fill the lumen or void.
